(19) United States
(12) Patent Application Publication
(10) Pub. No.: US 2024/0197510 A1
(43) Pub. Date: Jun. 20, 2024

(54) TECHNIQUES AND INSTRUMENTATION FOR THE REMOVAL OF RETAINED URETERAL STENTS

(71) Applicant: MDHA INVESTMENT PTY LTD, BRIGHTON (AU)

(72) Inventor: Dinesh AGARWAL, BRIGHTON (AU)

(21) Appl. No.: 18/556,975

(22) PCT Filed: Apr. 26, 2022
(86) PCT No.: PCT/AU2022/050377

§ 371 Date: Oct. 24, 2023

(30) Foreign Application Priority Data
Apr. 26, 2021 (AU) 2021901223

(51) Int. Cl. A61F 2/966; A61F 2/95

(52) U.S. Cl. CPC A61F 2/966; A61F 2002/9528

(57) ABSTRACT

A technique for the removal of a retained ureteral stent involves attaching a proximal end of the stent to a pulling member and pulling the proximal end of the stent through a lumen of an elongate device using the pulling member so that a distal end of the device advances along the stent up the ureter towards a blockage retaining the stent. As such, pressure applied by the distal end of the device in opposition to the stent pulled in respective tension removes the blockage.

### FIELD OF THE INVENTION

This invention relates generally to techniques and instrumentation for the removal of retained ureteral stents.

### BACKGROUND OF THE INVENTION

Ureteral stents are widely used in urologic practice to relieve the obstruction of the upper urinary tract and as a useful adjunct to various urological procedures.

One of the most common indications for a ureteral stent is the management of stone disease of upper urinary tract.

Ureteral stents are used for a temporary period and the duration therefor may vary depending upon the type of procedure. However, ureteral stents are sometimes used as a permanent measure to decompress the upper tract in patients who are not suitable for definitive procedures.

Patients with permanent stents require stent changing on a regular basis depending upon the type of stent and clinical indications.

However, ureteral stents sometimes become retained by a blockage caused by encrustation or even knotting. Patients with retained stents may pose a clinical challenge if the stents do not yield to simple cystoscopic procedure.

Patients with encrusted ureteral stents may require additional procedures such as endoscopic litholapexy, ureterorenoscopy, extracorporeal shock wave lithotripsy (ESWL), percutaneous nephrolithotomy (PCNL) or open surgery either alone or in combination depending upon the site and degree of encrustations.

There are no formal guidelines that exist as to the maximum safe indwelling time for ureteral stents with minimal risk of developing encrustations. The risk factors for stent encrustation may include prolonged indwelling time, stent composition, bacterial colonization, history of urolithiasis and pregnancy.

Treatment of the encrusted stent may depend upon the site and the degree of encrustation of the ureteric stents. Five grades of encrustations have been described depending upon the degree of calcifications. In Grade 1, only the bladder curl of stent is calcified. These type of retained stents can be removed after treating the bladder curl calcification by cystolitholapexy. In severe Grades, the upper curl and body of stent may be encrusted for varying degree. The cases with severe grades of calcification may require additional and more invasive procedures.

Blockage caused by knotting of ureteral stents is a rarer complication wherein, for example, a knot may form in the upper curl of the ureteral stent within the renal pelvis. It is speculated that excessive coil length may cause knot formation. Knotting may be sometimes addressed simply by pulling the knotted stent but which however poses the risk of ureteral avulsion. Alternatively, retrograde ureteroscopy and holmium laser fragmentation of the knot poses technical challenges in passing the ureteroscope past the retained stent. As such, more invasive procedures such as percutaneous approach and even open ureterotomy may be required.

The present invention seeks to provide a way to remove retained encrusted and/or knotted stents.

It is to be understood that, if any prior art information is referred to herein, such reference does not constitute an admission that the information forms part of the common general knowledge in the art, in Australia or any other country.

### SUMMARY OF THE DISCLOSURE

There is provided herein a retained ureteral stent removal technique comprising attaching a proximal end of the stent to a pulling member and pulling the proximal end of the stent through a lumen of an elongate device using the pulling member so that a distal end of the device advances along the stent up a ureter towards a blockage retaining the stent.

As such, pressure applied by the distal end of the device in opposition to the stent pulled in respective tension removes the blockage.

The present technique is a much simpler and less invasive technique as compared to existing techniques.

The proximal end of the stent may be pulled via an opening of the urethra. The proximal end of the stent may be exposed from the opening prior the proximal end of the stent being attached to the pulling member. The proximal end of the stent may be exposed from the opening prior the distal end of the device engaging the proximal end of the stent.

The distal end of the device may be advanced to the blockage. A distal end of the stent may be pulled through the distal end of the device whilst the device remains substantially in position. A distal end of the stent may be pulled through the proximal end of the device whilst the device remains substantially in position.

The blockage may be caused by encrustation of an exterior surface of the stent and wherein the pressure applied by the distal end of the device in opposition to the stent under relative tension dislodges the encrustation from the exterior surface of the stent. The stent may be pulled substantially free of encrustation through the distal end of the device.

The blockage may be caused by encrustation of an exterior surface of the distal end of the stent and wherein the pressure applied by the distal end of the device in opposition to the stent under relative tension causes at least some of the encrustation to enter the distal end of the device. The device may be removed from the ureter with at least some of the encrustation therein.

The device may comprise an outer sheath coaxially and slidably retaining an inner sheath therein and wherein the inner sheath may be pulled from the outer sheath to allow at least some of the encrustation to enter a distal end of the outer sheath. The inner sheath may be removed from the outer sheath with the outer sheath remaining substantially in position.

The blockage may be caused by knotting of the stent and wherein the pressure applied by the distal end of the device in opposition to the stent under relative tension unknots the knotting. A distal end of the stent may be pulled free of knotting through the distal end of the device.

The blockage may be caused by knotting of a distal end of the stent and wherein the pressure applied by the distal end of the device in opposition to the stent under tension tightens the knotting to decrease size of the knotting. The knotting of decreased size may be removed from the ureter with the device.

The blockage may be caused by knotting of a distal end of the stent and wherein the pressure applied by the distal end of the device in opposition to the stent under relative tension causes at least some of the knotting to enter the distal end of the device. The device may comprise an outer sheath slidably and coaxially retaining an inner sheath therein and wherein the inner sheath may be pulled from the outer sheath to allow at least some of the knotting to enter a distal end of the outer sheath. The outer sheath with at least some of the knotting therein may be removed from the ureter. The pressure applied by the distal end of the device in opposition to the stent under tension may tighten the knotting to decrease size of the knotting for entry into the outer sheath. The inner sheath may be removed from the outer sheath with the outer sheath remaining substantially in position.

The pulling member may comprise a flexible rod slidably passing through the device. A distal end of the flexible rod may comprise an attachment hole. A suture may attach the attachment hole to a proximal end of the stent. The suture may go through a proximal side drainage hole of the stent.

According to another aspect, there is provided retained ureteral stent removal instrumentation comprising a pulling member attachable to a proximal end of a stent and an elongate device having a lumen through which the pulling member may be pulled so that a distal end of the device advances along the stent up a ureter towards a blockage retaining the stent to remove the blockage. The device may comprise an outer sheath slidably and coaxially retaining an inner sheath therein.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows a technique of removing encrusted stents wherein FIG. 1A shows an encrusted ureteral stent, FIG. 1B illustrates the advancement of an elongate device along the encrusted ureteral stent; FIG. 1C illustrates the removal of the stent free of encrustation in an embodiment; FIG. 1D illustrates wherein the device comprises an outer sheath and an inner sheath and wherein at least some of the encrustation enters a distal end of the outer sheath in accordance with an embodiment; and FIG. 1E shows wherein a unitary device (i.e. without outer and inner sheaths) is used in accordance with an embodiment.

FIG. 2 shows a technique for removing knotted stents wherein FIG. 2A illustrates a ureteral stent retained by knotting; FIG. 2B illustrates the advancement of a device towards the knotting; FIG. 2C illustrates tension applied to the stent by the device to untie the knotting in accordance with an embodiment; FIG. 2D illustrates tension applied to the stent by the device to tighten the knotting to diminish the size of the knotting in accordance with an embodiment; and FIG. 2E illustrates utilisation of a device comprising an outer and inner sheath and wherein the inner sheath is retracted to allow at least a portion of the knotting to enter a distal end of the outer sheath in accordance with an embodiment;

FIG. 3A shows instrumentation for the removal of retained ureteral stents; and

FIG. 3B shows a flexible rod pulling member of the instrumentation in accordance an embodiment.

### DESCRIPTION OF EMBODIMENTS

A ureteral stent 101 may be retained within a ureter 102 or kidney by a blockage. The ureter 102 connects to the bladder 117. The ureteral stent 101 may comprise a plurality of side drainage holes 113 predominantly at distal and proximal portions of the stent 101.

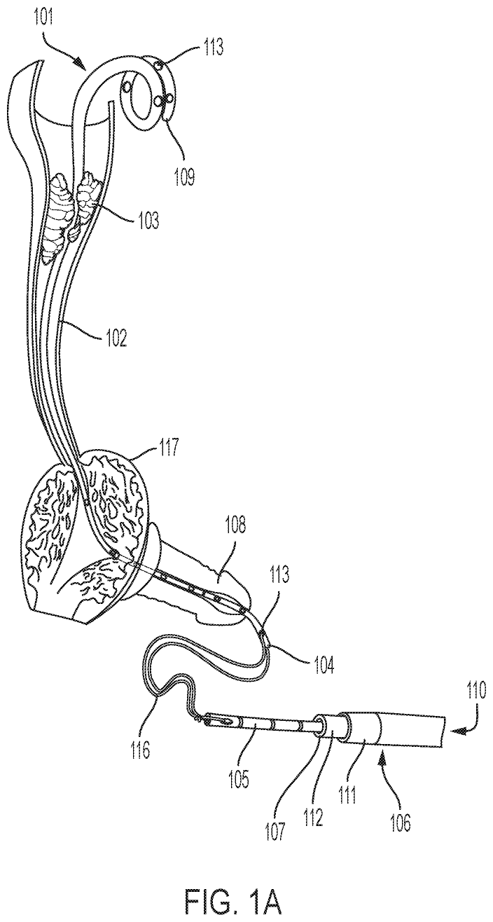

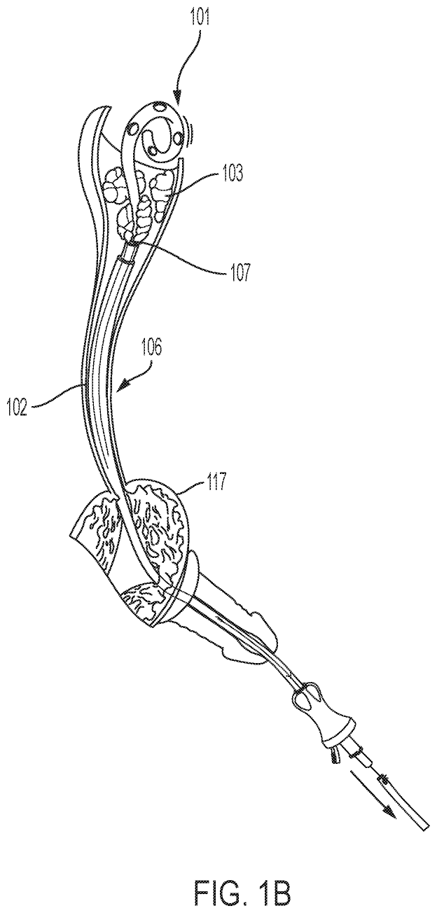

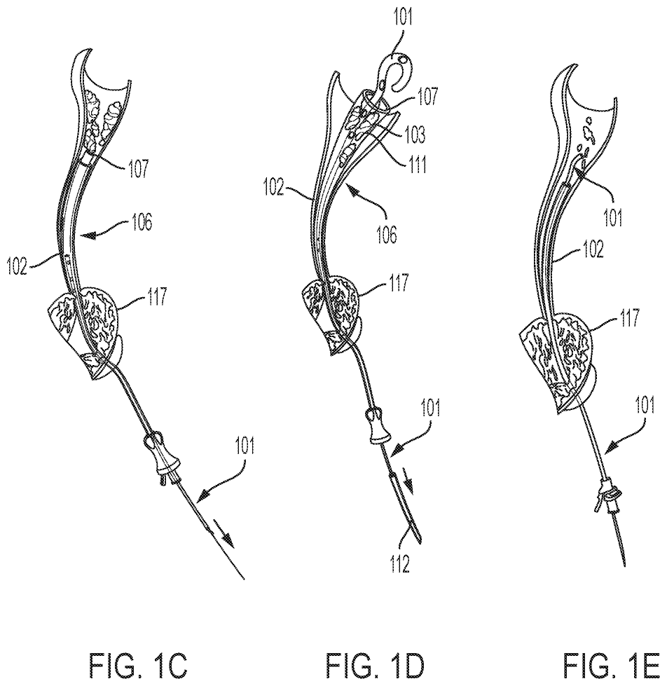

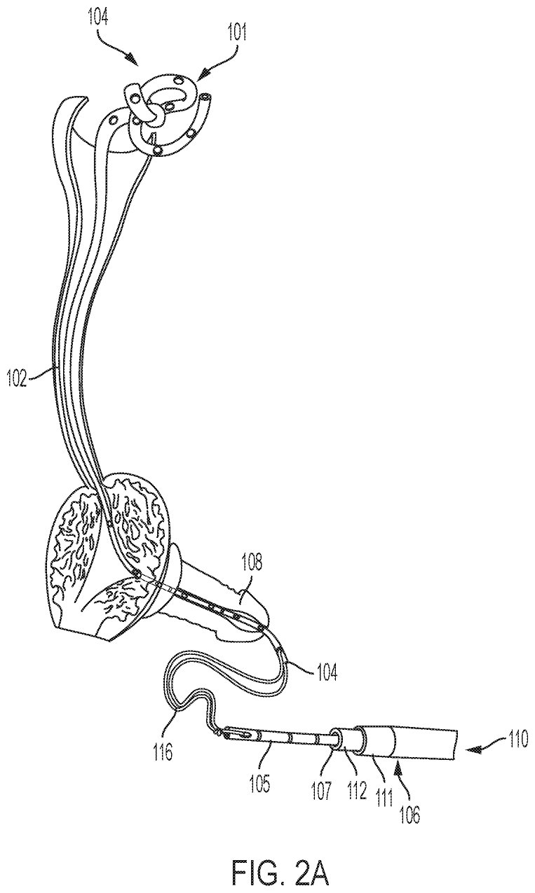

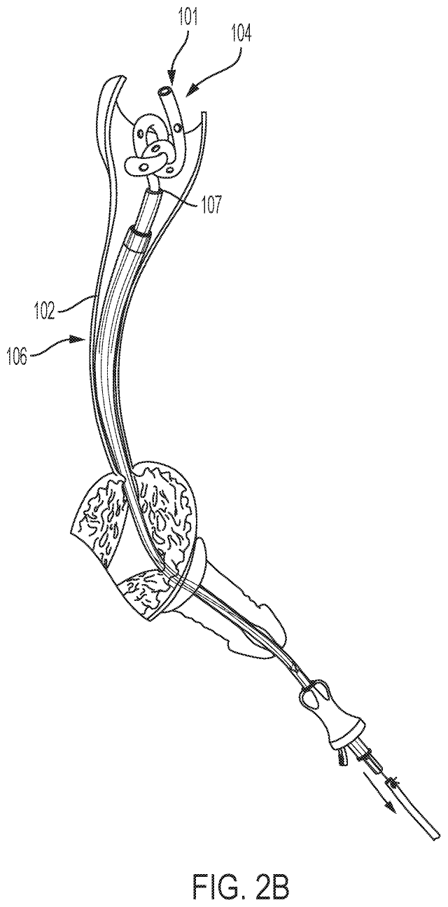

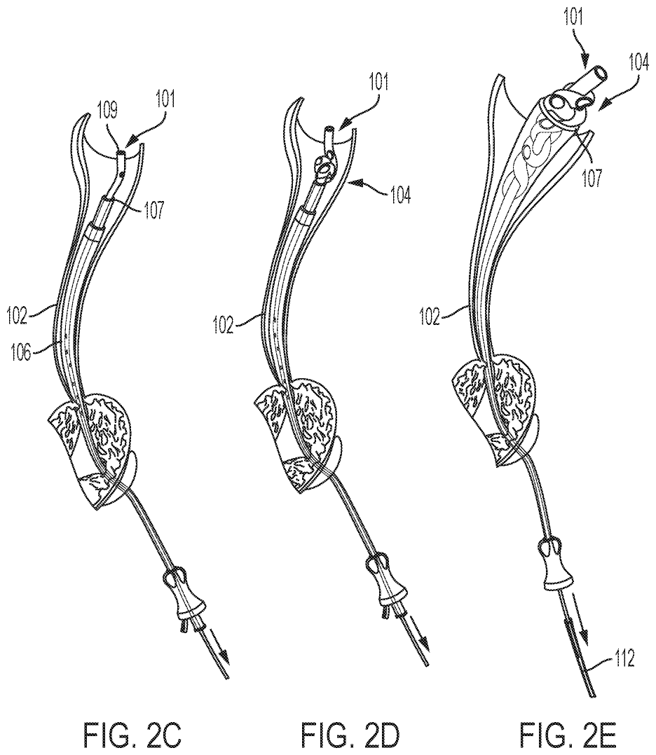

More specifically, FIG. 1A shows the ureteral stent 101 retained within the ureter 102 by encrustation 103 (such as urea mineral encrustation) whereas FIG. 2A shows the ureteral stent 101 retained by knotting 104.

A technique for removal of the retained ureteral stent 101 comprises attaching a proximal end 104 of the stent 101 to a pulling member 105 and pulling the proximal end 104 of the stent 101 through a lumen of an elongate device 106 using the pulling member 105 so that a distal end 107 of the device 106 advances along the stent 101 up the ureter 102 towards the blockage retaining the stent 101.

As such pressure applied by the distal end 107 of the device 106 in opposition to the stent 106 pulled in respective tension removes the blockage.

The proximal end 104 of the stent 101 may be pulled an opening of the urethra. FIGS. 1 and 2 show wherein the proximal end 104 of the stent 101 is pulled from the urinary meatus 108 of a male patient. The proximal end 104 of the stent 101 may be exposed from the opening of the urethra prior the proximal end 104 of the stent 101 being attached to the pulling member 105 or the distal end 107 of the device 106 engaging the proximal end 104 of the stent 101.

The distal end 107 of the device 106 may be advanced along the stent 101 to the site of the blockage whereafter a distal end 109 of the stent 101 is pulled through the distal end 107 of the device 106 whilst the device 106 remains substantially in position. The stent 101 may be pulled entirely from the device 106 wherein the distal end 109 of the stent 101 is pulled through a proximal end 110 of the device 106 whilst the device 106 remains substantially in position.

FIG. 1B shows wherein the blockage is caused by encrustation 103 of an exterior surface of the stent 101 and wherein pressure applied by the distal end 107 of the device 106 in opposition to the stent 101 under relative tension dislodges the encrustation 103 from the exterior surface of the stent 101. The stent 101 may be pulled free of encrustation 103 through the device 106 as is illustrated in FIG. 1C.

In some cases, at least a portion of the encrustation 103 may enter the distal end 107 of the device 106 so that at least a portion of the encrustation 103 may be removed with the device 106.

In embodiments, the device 106 may comprise an outer sheath 111 slidably and coaxially retaining an inner sheath 112 therein. The inner sheath 112 may assist in the advancement of the device 106 along the stent 101 or assist in the removal of encrustation 103 from the stent 101.

FIG. 1D shows wherein, once the device 106 is in position, the inner sheath 112 is pulled from the outer sheath 111 to allow at least some of the encrustation 103 to enter the distal end 107 of the device 106. As is illustrated, the inner sheath 112 may be removed from the outer sheath 111 with the outer sheath 111 remaining substantially in position.

As alluded to above, FIG. 2 shows wherein the blockage is caused by knotting 104 of the stent 101.

FIG. 2C illustrates the technique wherein pressure applied by the distal end 107 of the device 106 in opposition to the stent 101 under relative tension unknots the knotting 104. As such, the distal end 109 of the stent 101 may be pulled free of knotting 104 through the distal end 107 of the device 104.

FIG. 2D shows the technique wherein the pressure applied by the distal end 107 of the device 106 in opposition to the stent 101 under relative tension causes the knotting 104 to tighten to decrease the size of the knotting 104.

The decreased size knotting 104 may then be removed with the device 106.

FIG. 2E shows the embodiment wherein the device 106 comprises the outer sheath 111 coaxially and slidably retaining the inner sheath 112 therein. As is illustrated, the inner sheath 112 may be pulled from the outer sheath 111 to allow at least some of the knotting 104 to enter the distal end 107 of the outer sheath 111.

The inner sheath 112 may be removed with the outer sheath 111 remaining substantially in place.

Furthermore, the outer sheath 111 with at least some of the knotting 104 therein may be removed from the ureter 102.

The knotting 104 may be tightened to reduce the size thereof to allow at least a portion of the reduced size knotting 104 to enter the distal end 107 of the outer sheath 111.

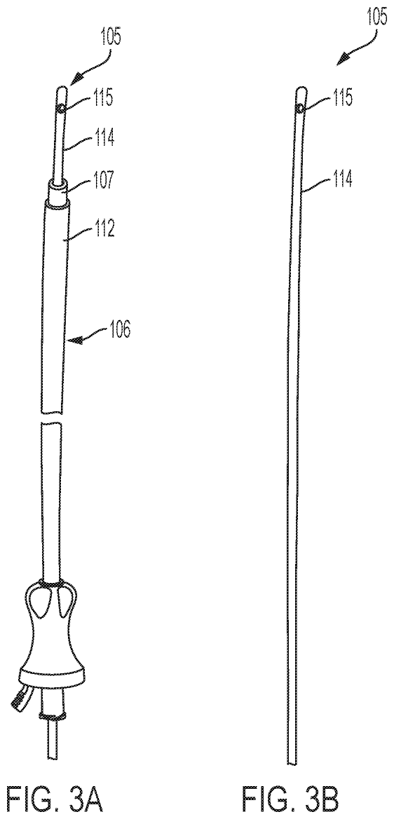

FIG. 3 shows wherein the pulling member 105 comprises a flexible rod 114, such as of plastic.

The rod 114 may comprise a distal attachment hole 115 therethrough. As is shown in FIGS. 1 and 2, a suture 116 through the attachment hole 115 may attach the distal portion 104 of the stent 101 to the rod 114. The suture 160 may be passed through a proximal drainage hole 113 of the stent 101.

The present technique should preferably be performed under fluoroscopic control with caution to avoid any ureteric injury. If there is any buckling of the device 106 or difficulty in advancing the device 106, the procedure should be abandoned.

Excessive force should be avoided while advancing the device 106. In the case of encrusted stents 101, once the distal end 107 of the device 106 is advanced up to the site of encrustation 103, gentle and intermittent pressure should be applied patiently as it may take few minutes to dislodge the encrustation 103.

The foregoing description, for purposes of explanation, used specific nomenclature to provide a thorough understanding of the invention. However, it will be apparent to one skilled in the art that specific details are not required in order to practise the invention. Thus, the foregoing descriptions of specific embodiments of the invention are presented for purposes of illustration and description. They are not intended to be exhaustive or to limit the invention to the precise forms disclosed as obviously many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following claims and their equivalents define the scope of the invention.

1. A retained ureteral stent removal technique comprising:
attaching a proximal end of the stent to a pulling member;
pulling the proximal end of the stent through a lumen of an elongate device using the pulling member and advancing a distal end of the elongate device along the stent up a ureter towards a blockage retaining the stent, wherein:
pressure applied by the distal end of the elongate device in opposition to the stent pulled in respective tension removes the blockage; and
the elongate device comprises an outer sheath coaxially and slidably retaining an inner sheath therein and wherein the inner sheath is pulled from the outer sheath to allow the blockage to enter a distal end of the outer sheath.

2. The technique as claimed in claim 1, wherein the proximal end of the stent is pulled via an opening of the urethra.

3. The technique as claimed in claim 2, wherein the proximal end of the stent is exposed from the opening of the urethra prior the proximal end of the stent being attached to the pulling member.

4. The technique as claimed in claim 2, wherein the proximal end of the stent is exposed from the opening of the urethra prior the distal end of the elongate device engaging the proximal end of the stent.

5. The technique as claimed in claim 1, wherein the distal end of the elongate device is advanced to the blockage.

6. The technique as claimed in claim 1, wherein a distal end of the stent is pulled through the distal end of the elongate device whilst the elongate device remains substantially in position.

7. The technique as claimed in claim 1, wherein a distal end of the stent is pulled through the proximal end of the elongate device whilst the elongate device remains substantially in position.

8. The technique as claimed in claim 1, wherein the blockage is caused by encrustation of an exterior surface of the stent and wherein the pressure applied by the distal end of the elongate device in opposition to the stent under relative tension dislodges the encrustation from the exterior surface of the stent.

9. The technique as claimed in claim 8, wherein the stent is pulled substantially free of encrustation through the distal end of the elongate device.

10. The technique as claimed in claim 1, wherein the blockage is caused by encrustation of an exterior surface of the distal end of the stent and wherein the pressure applied by the distal end of the elongate device in opposition to the stent under relative tension causes at least some of the encrustation to enter the distal end of the elongate device.

11. The technique as claimed in claim 10, wherein the elongate device is removed from the ureter with at least some of the encrustation therein.

12. The technique as claimed in claim 10, wherein the inner sheath is pulled from the outer sheath to allow at least some of the encrustation to enter a distal end of the outer sheath.

13. The technique as claimed in claim 12, wherein the inner sheath is removed from the outer sheath with the outer sheath remaining substantially in position.

14. The technique as claimed in claim 1, wherein the blockage is caused by knotting of the stent and wherein the pressure applied by the distal end of the elongate device in opposition to the stent under relative tension unknots the knotting.

15. The technique as claimed in claim 14, wherein a distal end of the stent is pulled free of knotting through the distal end of the elongate device.

16. The technique as claimed in claim 1, wherein the blockage is caused by knotting of a distal end of the stent and wherein the pressure applied by the distal end of the elongate device in opposition to the stent under tension tightens the knotting to decrease size of the knotting.

17. The technique as claimed in claim 16, wherein the knotting of decreased size is removed from the ureter with the elongate device.

18. The technique as claimed in claim 1, wherein the blockage is caused by knotting of a distal end of the stent and wherein the pressure applied by the distal end of the elongate device in opposition to the stent under relative tension causes at least some of the knotting enter the distal end of the elongate device.

19. The technique as claimed in claim 18, wherein the inner sheath is pulled from the outer sheath to allow at least some of the knotting to enter a distal end of the outer sheath.

20. The technique as claimed in claim 19, wherein the outer sheath with at least some of the knotting therein is removed from the ureter.

21. The technique as claimed in claim 19, wherein the pressure applied by the distal end of the elongate device in opposition to the stent under tension tightens the knotting to decrease size of the knotting for entry into the outer sheath.

22. The technique as claimed in claim 19, wherein the inner sheath is removed from the outer sheath with the outer sheath remaining substantially in position.

23. The technique as claimed in claim 1, wherein the pulling member comprises a flexible rod slidably passing through the elongate device.

24. The technique as claimed in claim 23, wherein a distal end of the flexible rod comprises an attachment hole.

25. The technique as claimed in claim 23, wherein a suture attaches the attachment hole to a proximal end of the stent.

26. The technique as claimed in claim 23, wherein the suture goes through a proximal side drainage hole of the stent.

27. (canceled)

28. (canceled)
